(19) United States
(12) Patent Application Publication
(10) Pub. No.: US 2017/0000421 A1
(43) Pub. Date: Jan. 5, 2017

(54) ARM-FASTENING DEVICE FOR MEASURING PULSE AND METHOD THEREOF

(71) Applicant: KOREA INSTITUTE OF ORIENTAL MEDICINE, Daejeon (KR)

(72) Inventors: Young Min KIM, Daejeon (KR); Chang Jin JUNG, Daejeon (KR); Jae Uk KIM, Daejeon (KR); Jong Yeol KIM, Daejeon (KR)

(21) Appl. No.: 15/125,934

(22) PCT Filed: Oct. 31, 2014
(86) PCT No.: PCT/KR2014/010325

§ 371 Date: Sep. 13, 2016

(30) Foreign Application Priority Data
Apr. 7, 2014 (KR) 10-2014-0041055

(51) Int. Cl. A61B 5/00; A61B 5/0205; A61B 5/107

(52) U.S. Cl. CPC A61B 5/702; A61B 5/1072; A61B 5/0205; A61B 5/7203; A61B 5/024

(57) ABSTRACT

An arm-fastening device may comprise: a guide part for elastically fastening a first recessed point in the lower part of the radius of an arm of a user and a second recessed point in the lower part of the ulna of the arm of the user; a measurement part for, when the arm is fastened, measuring the length of the arm, the length being from a reference surface to the point where the guide part fastens the first recessed point and the second recessed point; and a determination part for determining whether the arm is fastened according to the result of a comparison of the measured length of the arm with a reference length used for determining whether the arm is fastened.

### TECHNICAL FIELD

The present invention relates to an arm fastening method and device for measuring a pulse and, more particularly, to a method and device for comparing a length of an arm fastened by a guide portion to a reference length of the arm and controlling fastening of the arm.

### BACKGROUND ART

Oriental medicine diagnosis methods are classified into four types, inspection, auscultation-olfaction, interrogation, and palpation. A method of estimating a status of a disease by sensing a pulsation of a blood pulse wave belongs to a pulse diagnosis which is included in the palpation.

The pulse diagnosis may be based on various features of pulsation, for example, a pulse strength, a pulse depth, a pulse speed, and a pulse roughness. Thus, a technique for accurately measuring a pulse may be required to measure the features of pulsation.

A related art (Korean patent registration No. 1033416) discloses “Pulse diagnosis robot system and pulse diagnosis method using the same”. Specifically, a pulse sensor attached to a finger of the pulse diagnosis robot may extract a pulse signal and determine a pulse diagnosis result. Also, the pulse diagnosis method may be performed by assisting a pulse diagnosis for examining functions of internal organs to provide an accurate pulse diagnosis result.

A position of a pulse diagnosis device and a pulsating point may be set based on positions of a hand and an arm of a user every time that a user changes the positions the hand and the arm on the pulse diagnosis device. Also, a perspective of an operator may be reflected during the setting and thus, it is difficult to represent exactly the same process with respect to the user.

Accordingly, there is a desire for a device for fastening an arm of a user or a patient and providing a guide line in order to accurately measure a pulse.

### DISCLOSURE OF INVENTION

### Technical Solutions

According to an aspect of the present invention, there is provided an arm fastening device including a guide portion configured to flexibly fasten a first recessed point and a second recessed point, where the first recessed point is below a radius of an arm of a user and the second recessed point is below an ulna of the arm, a measurer configured to measure an arm length when the arm is fastened, the arm length being a length from a point at which the arm is fastened by the guide portion to a reference face, and a determiner configured to compare the measured arm length to a reference length used for determining whether the arm is fastened and determine whether the arm is fastened based on a comparison result.

The arm fastening device may further include a guide line providing portion configured to provide a guide line for measuring a pulse of the user when the determiner determines that the arm is fastened.

The guide line providing portion may be configured to select a guide line pattern from one or more guide line patterns and provide the selected guide line pattern.

The guide line providing portion may be configured to adjust an interval of the guide line based on the arm length.

The guide line providing portion may be configured to provide one or more sub-guide lines arranged in parallel.

An interval between the one or more sub-guide lines may be adjusted based on a distance between the one or more sub-guide lines and a change in a pulse wave of the user.

The guide portion may include a first guide portion configured to move on a surface of a side of the radius on the arm, detect the first recessed point, and fasten the detected first recessed point, and a second guide portion configured to move on a surface of a side of the ulna on the arm, detect the second recessed point, and fasten the detected second recessed point.

The measurer may be configured to compare a position of the first guide portion and a position of the second guide portion and measure a gradient of the guide portion relative to the reference face, and the determiner may be configured to determine whether the arm is fastened based on the measured gradient.

The determiner may include a calculator configured to calculate a first error between a first reference length and a first length from the first guide portion to the reference face, and calculate a second error between a second reference length and a second length from the second guide portion to the reference face.

The calculator may be configured to calculate a total error based on the first error and the second error and determine whether the arm is fastened.

The determiner may further include a controller configured to control the guide portion to refasten the first recessed point and the second recessed point when the total error is greater than or equal to a preset threshold.

The arm fastening device may further include a storage configured to store the reference length of the user.

The arm fastening device may further include a fastener configured to fasten an elbow of the user.

According to another aspect of the present invention, there is also provided an arm fastening method including flexibly fastening, by a guide portion, a first recessed point and a second recessed point, wherein the first recessed point is below a radius of an arm of a user and the second recessed point is below an ulna of the arm, measuring, by a measurer, an arm length when the arm is fastened, the arm length being a length from a point at which the arm is fastened by the guide portion to a reference face, and comparing, by a determiner, the measured arm length to a reference length used for determining whether the arm is fastened and determining whether the arm is fastened based on a comparison result.

The method may further include providing, by a guide line providing portion, a guide line for measuring a pulse of the user when the determiner determines that the arm is fastened.

The flexibly fastening may include moving, by a first guide portion, on a surface of a side of the radius on the arm, detecting the first recessed point, and fastening the first recessed point, and moving, by a second guide portion, on a surface of a side of the ulna on the arm, detecting the second recessed point, and fastening the second recessed point.

The method may further include calculating, by a calculator, a first error between a first reference length and a first length from the first guide portion to the reference face, and a second error between a second reference length and a second length from the second guide portion to the reference face.

The method may further include calculating, by the calculator, a total error based on the first error and the second error and determining whether the arm is fastened.

The method may further include controlling, by the determiner, the guide portion to refasten the first recessed point and the second recessed point when the total error is greater than or equal to a preset threshold.

According to still another aspect of the present invention, there is also provided a non-transitory computer-readable medium including a program for instructing a computer to perform an arm fastening method, wherein the method includes flexibly fastening, by a guide portion, a first recessed point and a second recessed point, where the first recessed point is below a radius of an arm of a user and the second recessed point is below an ulna of the arm, measuring, by a measurer, an arm length when the arm is fastened, the arm length being a length from a point at which the arm is fastened by the guide portion to a reference face, and comparing, by a determiner, the measured arm length to a reference length used for determining whether the arm is fastened and determining whether the arm is fastened based on a comparison result.

### BRIEF DESCRIPTION OF DRAWINGS

FIG. 1 is a block diagram illustrating an arm fastening device according to an embodiment of the present invention.

FIG. 2 is a block diagram illustrating a determiner of an arm fastening device according to an embodiment of the present invention.

FIG. 3 is a top view illustrating a guide portion fastening an arm of a user according to an embodiment of the present invention.

FIG. 4 is a view illustrating an example of providing a guide line on an arm of a user according to an embodiment of the present invention.

FIG. 5 illustrates a front view and a side view of a guide portion fastening an arm of a user according to an embodiment of the present invention.

FIG. 6 illustrates a fastener to fasten an elbow of a user according to an embodiment of the present invention.

FIG. 7 is a flowchart illustrating an arm fastening method according to an embodiment of the present invention.

FIG. 8 is a flowchart illustrating a method of determining whether an arm of a user is fastened according to an embodiment of the present invention.

### BEST MODE FOR CARRYING OUT THE INVENTION

Hereinafter, example embodiments will be described in detail with reference to the accompanying drawings. Like reference numerals refer to the like elements throughout.

The terms used in this specification were selected to include current, widely-used, general terms, in consideration of the functions of the present invention. However, the terms may represent different meanings according to the intentions of the skilled person in the art or according to customary usage, the appearance of new technology, etc.

In certain cases, a term may be one that was arbitrarily established by the applicant. In such cases, the meaning of the term will be defined in the relevant portion of the detailed description. As such, the terms used in the specification are not to be defined simply by the name of the terms but are to be defined based on the meanings of the terms as well as the overall description of the present invention.

Hereinafter, an arm fastening device and an arm fastening method will be described in detail.

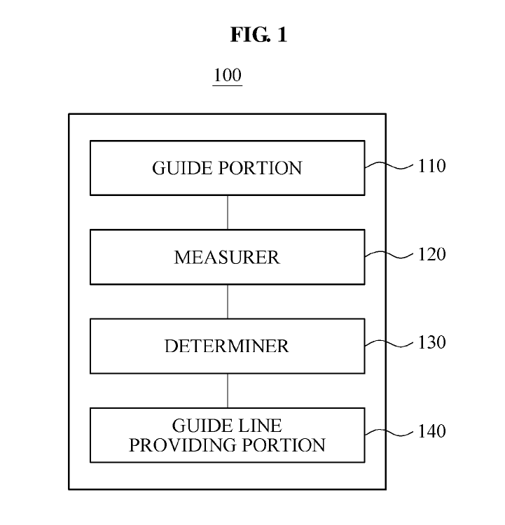

FIG. 1 is a block diagram illustrating an arm fastening device according to an embodiment of the present invention.

An arm fastening device 100 may include a guide portion 110, a measurer 120, and a determiner 130.

The guide portion 110 may fasten an arm of a user. Specifically, the guide portion 110 may fasten a first recessed point below a radius of the arm and a second recessed point below an ulna of the arm.

In detail, the radius and the ulna are protrusively formed in a portion of the arm extending from a wrist. The radius and the ulna respectively exist on both sides of the arm, for example, in a left portion and a right portion of a surface of the arm. Since the radius relatively protrudes from a surrounding area, the first recessed point may exist in a recessed area under the radius. Also, since the ulna relatively protrudes from a surrounding area, the second recessed point may exist in a recessed area under the ulna. Because the first recessed point and the second recessed point are relatively recessed from the surrounding areas, the first recessed point and the second recessed point may be suitable for fastening the arm and measuring a pulse.

The guide portion 110 may include a first guide portion and a second guide portion. The guide portion 110 may flexibly fasten the first recessed point and the second recessed point of the user.

The first guide portion and the second guide portion may be flexibly connected to an axis used to move the first and second guide portions. For example, the first guide portion and the second guide portion may be connected to the axis through a flexible body such as a spring. The spring is merely an example of the flexible body and thus, it is obvious to those skilled in the art that various types of flexible bodies are also applicable to the present disclosure. Through this, the arm may be flexibly fastened when the first guide portion and the second guide portion move on a surface of the arm.

The first guide portion may move on a a surface of a side of the radius on the arm, detect the first recessed point below the radius, and fasten the first recessed point. The second guide portion may move on a surface of a side of the ulna on the arm, detect the second recessed point below the ulna, and fasten the second recessed point.

The user may fasten the first recessed point and the second recessed point of the arm on the first guide portion and the second guide portion in person. Also, the first guide portion and the second guide portion may move and search for the first recessed point and the second recessed point to find the first recessed point and the second recessed point.

For example, the first guide portion and the second guide portion may detect a profile line in an x-axial direction while moving in a longitudinal direction of the arm, for example, in a y-axial direction. The determiner 130 may detect the first recessed point and the second recessed point based on the detected profile line.

The first guide portion may detect a recessed portion below the radius to be the first recessed point and fasten the first recessed point. To detect the first recessed point, a sensor may be attached to the first guide portion. In this example, the first guide portion may automatically move and detect the first recessed point using the sensor.

The second guide portion may detect a recessed portion below the ulna to be the second recessed point and fasten the second recessed point. To detect the second recessed point, a sensor may be attached to the second guide portion. In this example, the second guide portion may automatically move and detect the second recessed point using the sensor.

When the arm is fastened by the first guide portion and the second guide portion, the measurer may measure an arm length indicating a length from a reference face to a point fastened by the guide portion. Here, the reference face may be, for example, a face supporting an elbow of the user. Also, the arm length may be, for example, a length from the reference face to a predetermined point on the arm.

The user may fasten the arm in a sitting posture. For example, the arm may be fastened at an angle of approximately 110 degrees between a line on a shoulder and the elbow and a line on the elbow and a wrist.

Also, the user may position the elbow on a fastener in the sitting posture and make a motion that the wrist is fastened by the guide portion.

The determiner 130 may determine whether the arm is fastened on the arm fastening device 100. The determiner 130 may compare the measured arm length to a reference length for determining whether the arm is fastened, and determine whether the arm is fastened based on a comparison result.

Here, the reference length may be a length used to be a reference for determining whether the arm is fastened. The reference length may be previously stored in the arm fastening device 100. The reference length may include a first reference distance indicating a length from the reference face to the first guide portion and a second reference distance indicating a length from the reference face to the second guide portion.

For example, the arm fastening device 100 may previously store or register the first reference length used to be a reference for accurately measure a pulse of a first user in a storage of the arm fastening device 100. When the first user fastens an arm on the arm fastening device 100 to measure a pulse, the arm fastening device 100 may compare the measured arm length to the first reference length and determine whether the arm is fastened. Thereafter, when the same user is to measure a pulse again, the arm fastening device 100 may quickly and accurately measure the pulse without need to perform an initial setting of a measurement environment.

In an example, the arm fastening device 100 may further include a storage configured to store the reference length of the user. The storage may store a reference length for each of a plurality of users. Also, the storage may store a reference value used as a reference for determining whether the arm is fastened. Thus, as well as the reference length, various values for determining whether the arm is fastened may also be stored in the storage.

Here, the storage of the arm fastening device 100 may be implemented as an external device separate from the arm fastening device 100, and may also be included in the arm fastening device 100. Accordingly, as well as an example in which the storage is included in the arm fastening device 100, an example in which the storage is implemented physically external to the arm fastening device 100 is also within the scope of the present disclosure.

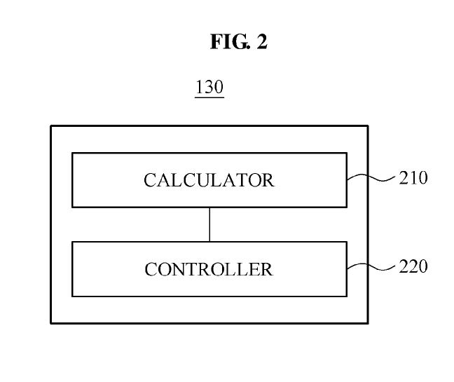

The determiner 130 may determine whether the arm is fastened based on a first error and a second error. Here, the first error may be a difference between a first length from the first guide portion to a reference face and a first reference length. The second error may be a difference between a second length from the second guide portion to the reference face and a second reference length. The first error and the second error will be also described with reference to FIG. 2.

Also, the determiner 130 may control the guide portion 110 to refasten the arm when an error is outside a predetermined error range. Related descriptions will be also provided with reference to FIG. 2.

In an example, the arm fastening device 100 may further include a guide line providing portion 140. The guide line providing portion 140 may provide a guide line on the arm of the user. The guide line may be provided to measure a pulse of the user. In this example, the guide line may be a line for detecting Cun, Guan, and Chi of the user. The line for detecting Cun, Guan, and Chi is described as an example and thus, the guide line providing portion 140 may provide various types of guide line patterns.

The guide line providing portion 140 may select a guide line pattern from one or more guide line patterns and provide the selected guide line pattern. Here, the guide line pattern may have, for example, a straight line form and a curved line form. The guide line providing portion 140 may select and provide the guide line pattern based on the arm length. For example, when the arm length is relatively long, the guide line providing portion 140 may increase a distance between sub-guide lines and provide the guide line pattern. Also, the guide line pattern may be determined in consideration of body conditions to accurately detect Cun, Guan, and Chi of a body.

The guide line providing portion 140 may provide one or more sub-guide lines arranged in parallel. An interval between sub-guide lines may be adjusted based on a distance between the sub-guide lines and a change in a pulse wave of the user.

When the arm is fastened, the arm fastening device 100 may provide a guide line. The interval between sub-guide lines may be proportional to the arm length. Here, the arm length may be, for example, a length from a point at which the guide portion fastens the arm to the reference face. The point may be, for example, a position of the first guide portion fastened on the first recessed point below the radius of the arm, and the second guide portion fastened on the second recessed point below the ulna of the arm.

The arm length may be, for example, an average value of a first length and a second length. The first length may be a length from the first recessed point to the reference face. The second length may be a length from the second recessed point to the reference face.

FIG. 2 is a block diagram illustrating a determiner of an arm fastening device according to an embodiment of the present invention.

A determiner of an arm fastening device may include a calculator 210 and a controller 220.

The calculator 210 may calculate a first error and a second error. The first error may be, for example, a difference value between a first length from a first guide portion to a reference face and a first reference length. The second error may be, for example, a difference value between a second length from a second guide portion to the reference face and a second reference length.

In an example, the first reference length may be stored or registered as Rleft in the arm fastening device, and the second reference length may be stored or registered as Rright in the arm fastening device. When a user positions an arm on the arm fastening device, a measurer may measure the first length and the second length. The first length may be represented as Cleft, and the second length may be represented as Cright.

The first error may be obtained by “Rleft−Cleft”, and the second error may be obtained by “Rright−Cright”. Whether the arm is fastened may be determined based on a preset threshold. A calculator may calculate a total error based on the first error and the second error. When the total error corresponding to a value calculated according to Equation 1 is less than the threshold, the calculator 210 may determine that the arm is fastened. When the total error is greater than or equal to the threshold, the calculator 210 may determine that the arm is not fastened. Equation 1 may be expressed as below.

(Rleft−Cleft)²+(Rright−Cright)²<Threshold [Equation 1]

The controller 220 may control a guide portion to refasten the arm in an unfastened state. The controller 220 may control the guide portion such that a first recessed point and a second recessed point are fastened. Alternatively, the controller 220 may control such that a feedback indication is output to the user to refasten the arm.

Hereinafter, various operations and applications performed by an arm fastening device will be described. Although one element, for example, a guide portion, a measurer, a guide line providing portion, a calculator, and a controller is not specified, contents of an extent that can be expected and understood clearly by those skilled in the art to which the present disclosure pertains may be understood as an ordinary implementation and thus, the scope of the present invention is not intended to be limited by the terms or physical/logical structures of the specific configurations.

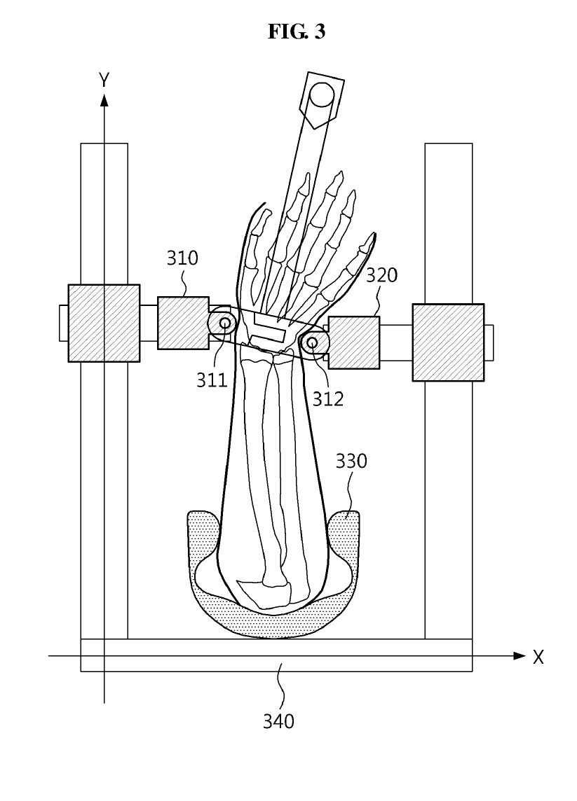

FIG. 3 is a top view illustrating a guide portion fastening an arm of a user according to an embodiment of the present invention.

An arm fastening device may fasten an arm of a user. The arm fastening device may user a guide portion to fasten a first recessed point below a radius of the arm and a second recessed point below an ulna of the arm.

The guide portion may include a first guide portion 311 and a second guide portion 321. The first guide portion 311 may move on a surface of a side of the radius to detect and fasten a first recessed point 312 below the radius. The second guide portion 321 may move on a surface of the ulna to detect and fasten a second recessed point 322 below the ulna.

A measurer may measure an arm length corresponding to a length from a point at which the guide portion fastens the arm to a reference face 340. The measurer may measure a first arm length and a second arm length. The first arm length may be a vertical length between the first recessed point and the reference face 340. The second arm length may be a vertical length between the second recessed point and the reference face 340.

In an example, the measurer may represent the first recessed point and the second recessed point by coordinates. For example, the first recessed point may be represented by coordinates (X1, Y1), and the second recessed point may be represented by coordinates (X2, Y2). Here, Y1 and Y2 may be the arm length.

A determiner may compare the measured arm length to a reference length for determining whether the arm is fastened, and determine whether the arm is fastened based on a comparison result. In this example, the reference length for determining whether the arm is fastened may be previously stored in a storage of the arm fastening device.

When the measured arm length is compared to the reference length, and when a total error is less than a preset threshold, the determiner may determine that the arm is fastened.

In this example, when the total error is greater than or equal to the threshold, the determiner may determine that the arm is not fastened and control the guide portion to refasten the arm. The guide portion may be controlled manually and automatically. When the guide portion is automatically controlled, the guide portion may include a sensor. The sensor may detect the first recessed point below the radius and the second recessed point below the ulna.

In an example, the measurer may compare a position of the first guide portion 311 to a position of the second guide portion 321, and measure a gradient of the guide portion relative to the reference face 340. The position of the first guide portion 311 may correspond to a point at which the first guide portion 311 fastens the first recessed point. The position of the second guide portion 321 may correspond to a point at which the second guide portion 321 fastens the second recessed point. The first recessed point may be represented by the coordinates (X1, Y1), and the second recessed point may be represented by the coordinates (X2, Y2). The gradient of the guide portion relative to the reference face 340 may be expressed as shown in Equation 2.

$$Gradient = \frac{Y 2 - Y 1}{X 2 - X 1} \qquad [ Equation 2 ]$$

The determiner may compare the measured gradient to a reference gradient for determining whether the arm is fastened, and determine whether the arm is fastened based on a comparison result. Here, the reference gradient may be previously stored in the storage.

When the measured gradient is compared to the reference gradient, and when the total error is less than the preset threshold, the determiner may determine that the arm is fastened.

In this example, when the total error is greater than or equal to the threshold, the determiner may determine that the arm is not fastened and control the guide portion to refasten the arm.

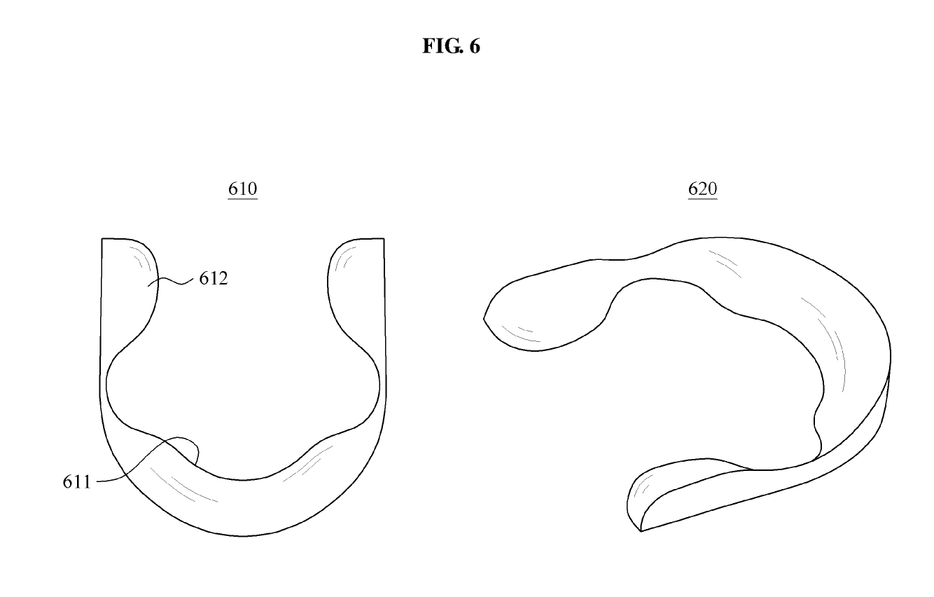

In an example, the arm fastening device may include a fastener 330 configured to fasten an elbow of the user. The fastener will be also described with reference to FIG. 6.

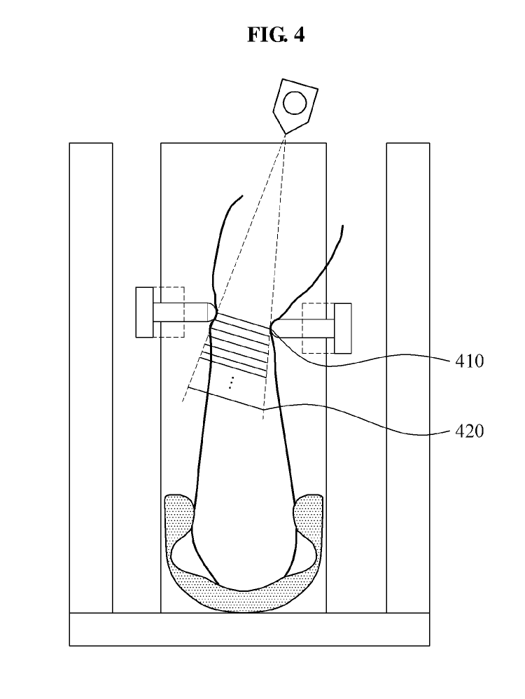

FIG. 4 is a view illustrating an example of providing a guide line on an arm of a user according to an embodiment of the present invention.

An arm fastening device may include a guide line providing portion.

When a determiner determines that an arm of a user is fastened, the guide line providing portion may provide a guide line for measuring a pulse of the user.

The guide line providing portion may provide a guide line 410 on a first recessed point of a lower portion of a radius and a second recessed point of a lower portion of an ulna.

In this example, the guide line providing portion may provide a guide line pattern from one or more guide line patterns and provide the selected guide line pattern.

The guide line pattern may correspond to at least one of a straight line form and a curved line form. For example, the guide line providing portion may provide one or more sub-guide line lines arranged in parallel. A form of guide line pattern is not limited to the foregoing example and thus, the guide line pattern may be provided in various forms.

In an example, the guide line providing portion may provide a plurality of sub-guide lines. The guide line providing portion may adjust an interval between the sub-guide lines based on an arm length of the user. Hereinafter, “adjust/adjusting the interval between the sub-guide lines” will be also expressed as “adjust/adjusting or set/setting a guide line interval”. For example, the guide line interval may be adjusted based on a position of a first guide portion and a position of a second guide portion. As described with reference to FIG. 3, the first recessed point may be represented by coordinates (X1, Y1), and the second recessed point may be represented by coordinates (X2, Y2). In this example, Y1 and Y2 may be the arm length and thus, the guide line interval may be set proportionally to an average of Y1 and Y2.

In another example, the guide line interval may be set based on a standard interval. Here, the standard interval may be set by measuring arm lengths of a plurality of users. The standard interval may represent a relationship between the arm length and the sub-guide line. The standard interval may be previously stored in a storage.

When the measurer measures the arm length of the user, and when the determiner compares the measured arm length to a reference length and determines that the arm is fastened, the guide line providing portion may provide a guide line having a standard interval corresponding to the arm length.

In still another example, an interval having a meaningful difference in pulse wave may be obtained by monitoring a change in the guide line interval and a change in a pulse wave, and the interval may be set as the standard interval. In this example, the interval may be an interval corresponding to, for example, Cun, Guan, and Chi.

Also, the guide line interval may be adjusted using a statistical correction constant based on at least one of a gender, an age, a muscle mass, and a body mass index (BMI).

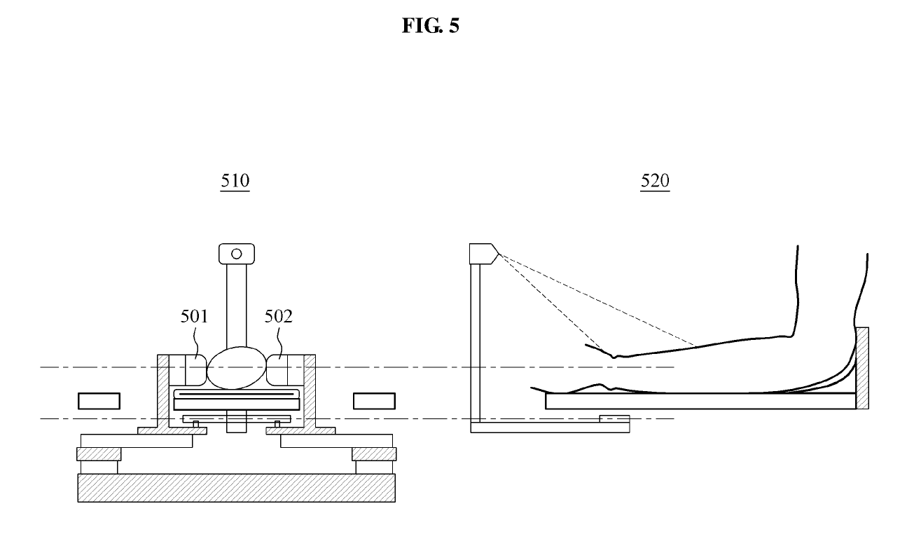

FIG. 5 illustrates a front view and a side view of a guide portion fastening an arm of a user according to an embodiment of the present invention.

A part 510 illustrates a front view of an arm fastening device on which a user fastens an arm. A first guide portion 501 may fasten a first recessed point below a radius of the arm. A second guide portion 502 may fasten a second recessed point below an ulna of the arm.

A part 520 illustrates a side view of the arm fastening device on which the user fastens the arm. When the arm is fastened, a guide line providing portion may provide a guide line on the arm as shown in the part 520.

FIG. 6 illustrates a fastener to fasten an elbow of a user according to an embodiment of the present invention.

In an example, an arm fastening device may further include a fastener. The fastener may fasten an elbow of a user. A part 610 illustrates a top view of the fastener. A portion 611 may be in contact with the elbow. A portion 612 may be in contact with a side of an arm of the user. Since a size of an arm varies by individuals, the portion 612 may be formed of a flexible material. A part 620 illustrates a side view of the fastener.

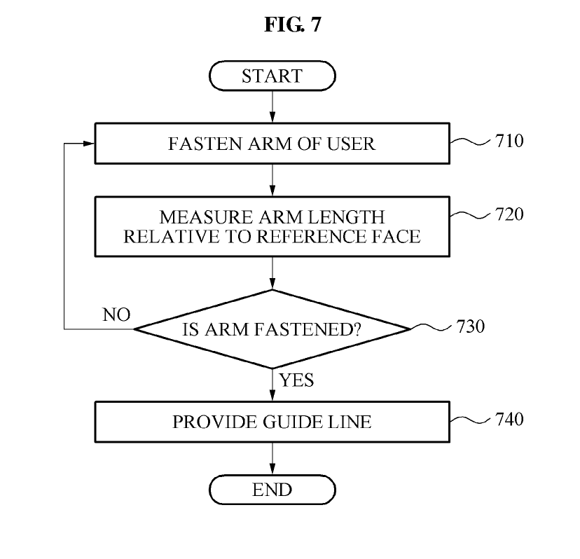

FIG. 7 is a flowchart illustrating an arm fastening method according to an embodiment of the present invention.

In operation 710, a guide portion may fasten an arm of a user. The guide portion may fasten a first recessed point below a radius of the arm and a second recessed point below an ulna of the arm. The guide portion may flexibly fasten the arm.

The guide portion may include a first guide portion and a second guide portion. The first guide portion may move on a surface of a side of the radius on the arm to detect and fasten the first recessed point. The second guide portion may move on a surface of a side of the ulna on the arm to detect and fasten the second recessed point.

In operation 720, a measurer may measure an arm length relative to a reference face. The arm length may be, for example, a vertical distance between a point at which the guide portion fastens the arm and the reference face. Here, the reference face may be a face in contact with an elbow of the user.

The measurer may measure a first length from the first guide portion to the reference face and a second length from the second guide portion to the reference face.

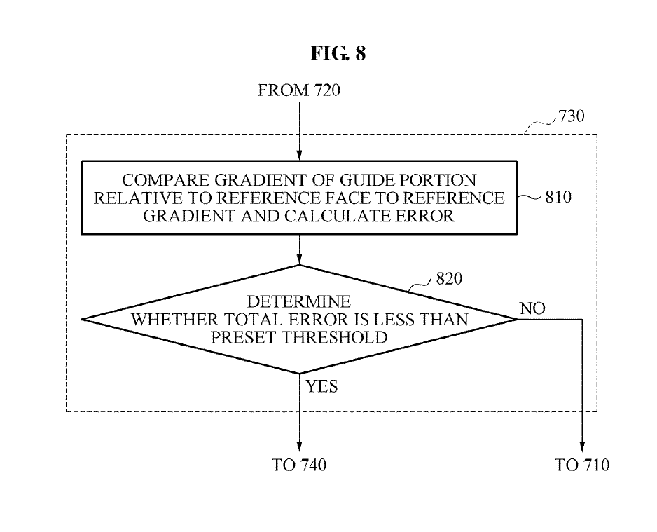

In operation 730, a determiner may determine whether the arm is fastened. The determiner may compare the measured arm length to a reference length used for determining whether the arm is fastened and determine whether the arm is fastened based on a comparison result. A determination whether the arm is fastened will be also described with reference to FIG. 8.

When the determiner determines that the arm is not fastened, the guide portion may be controlled to refasten the arm. In this example, a procedure may be performed by returning to operation 710. When the determiner determines that the arm is fastened, operation 740 may be performed.

In operation 740, a guide line providing portion may provide a guide line on the arm.

FIG. 8 is a flowchart illustrating a method of determining whether an arm of a user is fastened according to an embodiment of the present invention.

In an example, an operation of determining whether an arm of a user is fastened may include operation 810 and operation 820.

In operation 810, an arm length relative to a reference face may be compared to a reference length and an error may be calculated.

A calculator may calculate a first error between a first length and a first reference length, and a second error between a second length and a second reference length. The calculator may calculate a total error based on the first error and the second error. Also, when the total error is less than a preset threshold, the calculator may determine that the arm is fastened. When the total error is greater than or equal to the threshold, the calculator may determine that the arm is not fastened. The descriptions of the total error provided with reference to Equation 1 are also applicable here.

In another example, in operation 810, a gradient of a guide portion relative to the reference face may be compared to a reference gradient and the error may be calculated.

The calculator may compare a position of a first guide portion to a position of a second guide portion and perform a gradient calculation. The descriptions of the gradient calculation provided with reference to FIG. 3 are also applicable here.

In operation 820, whether the total error is less than a preset threshold may be determined. In response to a determination that the total error is less than the threshold, the calculator may control a guide line providing portion to provide a guide line on the arm of the user. In response to a determination that the total error is greater than or equal to the threshold, the calculator may control the guide portion to refasten the arm of the user.

When the total error is less than the threshold, operation 740 may be performed. When the total error is greater than or equal to the threshold, operation 710 may be performed.

The units described herein may be implemented using hardware components, software components, and combinations thereof. For example, the hardware components may include microphones, amplifiers, band-pass filters, audio to digital convertors, and processing devices. A processing device may be implemented using one or more general-purpose or special purpose computers, such as, for example, a processor, a controller and an arithmetic logic unit, a digital signal processor, a microcomputer, a field programmable array, a programmable logic unit, a microprocessor or any other device capable of responding to and executing instructions in a defined manner.

The processing device may run an operating system (OS) and one or more software applications that run on the OS. The processing device also may access, store, manipulate, process, and create data in response to execution of the software.

For purpose of simplicity, the description of a processing device is used as singular; however, one skilled in the art will appreciated that a processing device may include multiple processing elements and multiple types of processing elements. For example, a processing device may include multiple processors or a processor and a controller. In addition, different processing configurations are possible, such a parallel processors.

The software may include a computer program, a piece of code, an instruction, or some combination thereof, for independently or collectively instructing or configuring the processing device to operate as desired.

Software and data may be embodied permanently or temporarily in any type of machine, component, physical or virtual equipment, computer storage medium or device, or in a propagated signal wave capable of providing instructions or data to or being interpreted by the processing device. The software also may be distributed over network coupled computer systems so that the software is stored and executed in a distributed fashion. In particular, the software and data may be stored by one or more computer readable recording mediums

The methods according to the above-described embodiments may be recorded, stored, or fixed in one or more non-transitory computer-readable media that includes program instructions to be implemented by a computer to cause a processor to execute or perform the program instructions. The media may also include, alone or in combination with the program instructions, data files, data structures, and the like. The program instructions recorded on the media may be those specially designed and constructed, or they may be of the kind well-known and available to those having skill in the computer software arts.

Examples of non-transitory computer-readable media include magnetic media such as hard disks, floppy disks, and magnetic tape; optical media such as CD ROM discs and DVDs; magneto-optical media such as optical discs; and hardware devices that are specially configured to store and perform program instructions, such as read-only memory (ROM), random access memory (RAM), flash memory, and the like.

Examples of program instructions include both machine code, such as produced by a compiler, and files containing higher level code that may be executed by the computer using an interpreter.

The described hardware devices may be configured to act as one or more software modules in order to perform the operations and methods described above, or vice versa.

While this disclosure includes specific examples, it will be apparent to one of ordinary skill in the art that various changes in form and details may be made in these examples without departing from the spirit and scope of the claims and their equivalents. Descriptions of features or aspects in each example are to be considered as being applicable to similar features or aspects in other examples. Suitable results may be achieved if the described techniques are performed in a different order, and/or if components in a described system, architecture, device, or circuit are combined in a different manner, and/or replaced or supplemented by other components or their equivalents.

Therefore, the scope of the disclosure is defined not by the detailed description, but by the claims and their equivalents, and all variations within the scope of the claims and their equivalents are to be construed as being included in the disclosure.

What is claimed is:

1. An arm fastening device comprising:
a guide portion configured to flexibly fasten a first recessed point and a second recessed point, where the first recessed point is below a radius of an arm of a user and the second recessed point is below an ulna of the arm;
a measurer configured to measure an arm length when the arm is fastened, the arm length being a length from a point at which the arm is fastened by the guide portion to a reference face; and
a determiner configured to compare the measured arm length to a reference length used for determining whether the arm is fastened and determine whether the arm is fastened based on a comparison result.

2. The arm fastening device of claim 1, further comprising:
a guide line providing portion configured to provide a guide line for measuring a pulse of the user when the determiner determines that the arm is fastened.

3. The arm fastening device of claim 2, wherein the guide line providing portion is configured to select a guide line pattern from one or more guide line patterns and provide the selected guide line pattern.

4. The arm fastening device of claim 3, wherein the guide line providing portion is configured to adjust an interval of the guide line based on the arm length.

5. The arm fastening device of claim 2, wherein the guide line providing portion is configured to provide one or more sub-guide lines arranged in parallel.

6. The arm fastening device of claim 5, wherein an interval between the one or more sub-guide lines is adjusted based on a distance between the one or more sub-guide lines and a change in a pulse wave of the user.

7. The arm fastening device of claim 1, wherein the guide portion includes:
a first guide portion configured to move on a surface of a side of the radius on the arm, detect the first recessed point, and fasten the detected first recessed point; and
a second guide portion configured to move on a surface of a side of the ulna on the arm, detect the second recessed point, and fasten the detected second recessed point.

8. The arm fastening device of claim 7, wherein the measurer is configured to compare a position of the first guide portion and a position of the second guide portion and measure a gradient of the guide portion relative to the reference face, and
the determiner is configured to determine whether the arm is fastened based on the measured gradient.

9. The arm fastening device of claim 7, wherein the determiner includes a calculator configured to calculate a first error between a first reference length and a first length from the first guide portion to the reference face, and calculate a second error between a second reference length and a second length from the second guide portion to the reference face.

10. The arm fastening device of claim 9, wherein the calculator is configured to calculate a total error based on the first error and the second error and determine whether the arm is fastened.

11. The arm fastening device of claim 10, wherein the determiner further includes a controller configured to control the guide portion to refasten the first recessed point and the second recessed point when the total error is greater than or equal to a preset threshold.

12. The arm fastening device of claim 1, further comprising:
a storage configured to store the reference length of the user.

13. The arm fastening device of claim 1, further comprising:
a fastener configured to fasten an elbow of the user.

14. An arm fastening method comprising:
flexibly fastening, by a guide portion, a first recessed point and a second recessed point, wherein the first recessed point is below a radius of an arm of a user and the second recessed point is below an ulna of the arm;
measuring, by a measurer, an arm length when the arm is fastened, the arm length being a length from a point at which the arm is fastened by the guide portion to a reference face; and
comparing, by a determiner, the measured arm length to a reference length used for determining whether the arm is fastened and determining whether the arm is fastened based on a comparison result.

15. The method of claim 14, further comprising:
providing, by a guide line providing portion, a guide line for measuring a pulse of the user when the determiner determines that the arm is fastened.

16. The method of claim 14, wherein the flexibly fastening includes:
moving, by a first guide portion, on a surface of a side of the radius on the arm, detecting the first recessed point, and fastening the first recessed point; and
moving, by a second guide portion, on a surface of a side of the ulna on the arm, detecting the second recessed point, and fastening the second recessed point.

17. The method of claim 16, further comprising:
calculating, by a calculator, a first error between a first reference length and a first length from the first guide portion to the reference face, and a second error between a second reference length and a second length from the second guide portion to the reference face.

18. The method of claim 17, further comprising:
calculating, by the calculator, a total error based on the first error and the second error and determining whether the arm is fastened.

19. The method of claim 18, further comprising:
controlling, by the determiner, the guide portion to refasten the first recessed point and the second recessed point when the total error is greater than or equal to a preset threshold.

20. A non-transitory computer-readable medium comprising a program for instructing a computer to perform an arm fastening method, wherein the method includes:
flexibly fastening, by a guide portion, a first recessed point and a second recessed point, where the first recessed point is below a radius of an arm of a user and the second recessed point is below an ulna of the arm;
measuring, by a measurer, an arm length when the arm is fastened, the arm length being a length from a point at which the arm is fastened by the guide portion to a reference face; and
comparing, by a determiner, the measured arm length to a reference length used for determining whether the arm is fastened and determining whether the arm is fastened based on a comparison result.
